(19) United States
(12) Patent Application Publication
(10) Pub. No.: US 2007/0167903 A1
(43) Pub. Date: Jul. 19, 2007

(54) Nervous Tissue Electrode Device

(75) Inventors: Juan Sanchez-Ramos, Tampa, FL (US); Richard Heller, Temple Terrace, FL (US); Mark J. Jaroszeski, Tampa, FL (US)

Correspondence Address: SMITH HOPEN, PA, 180 PINE AVENUE NORTH, OLDSMAR, FL, 34677, US

(73) Assignee: UNIVERSITY OF SOUTH FLORIDA, Tampa, FL (US)

(21) Appl. No.: 11/160,180

(22) Filed: Jun. 13, 2005

Related U.S. Application Data
(60) Provisional application No. 60/521,651, filed on Jun. 11, 2004.

(51) Int. Cl. A61N 1/30

(52) U.S. Cl. 604/21

(57) ABSTRACT

In accordance with the present invention is provided a novel tri-pronged electrode that produces minimal damage to deep brain or spinal cord regions. The apparatus and associated method of the present invention provides for a single insertion of a novel probe effective for both delivery of DNA and delivery of therapeutic electroporating current, thereby minimizing the potential damage to the nervous system.

### CROSS REFERENCE TO RELATED APPLICATIONS

This application claims priority to currently pending U.S. Provisional Patent Application No. 60/521,651, filed Jun. 11, 2004.

### BACKGROUND OF INVENTION

Delivery of genes for the effective therapy of brain and spinal cord diseases generally requires a viral vector, however, recent development in the field of gene therapy has provided for in vivo electroporation for the delivery of therapeutic genes without the need for a viral vector.

Current electroporation techniques known in the art require two passages of probes into the subject tumor. The first probe is typically a needle, placed stereotaxically, for the delivery of the DNA. The second probe is the passage of a pair of electrodes for the delivery of the required current for electroporation.

What is needed in the art is a device capable of DNA delivery and electroporation utilizing a single probe passage into the tissue. Such a device would minimize the potential damage to the nervous system of a patient.

### SUMMARY OF INVENTION

In accordance with the present invention is provided a novel tri-pronged electrode for the delivery of DNA and electroporation that produces minimal damage to deep brain or spinal cord regions.

In a particular embodiment, the present invention provides a device for the delivery of genes into tissue, the device including a cast polymer support having an aperture, at least two spaced apart needle electrodes secured within the cast polymer support and positioned to surround the aperture and an axially adjustable injection cannula positioned in the aperture, the axial position of the axially adjustable injection cannula adjustable along the axis of the at least two spaced apart needle electrodes. The cast polymer support may be cylindrical or of any other of a variety of geometric configurations known in the art.

Additionally, after the injection cannula has been positioned at a selected axial position, the cannula may be secured relative to the support utilizing an injection cannula fastener.

The device may additionally include an electric pulse generator for applying pulses to the at least two needle electrodes for electroporation of cells between said electrodes. With this embodiment, the needle electrodes and the injection cannula are introduced to the tissue and the cannula is adjusted along the length of the needle electrodes to any of a variety of positions for administration of the DNA. The pulses are then applied to the needle electrodes for electroporation of the cells of the tissue for effective treatment.

The apparatus and associated method of the present invention provides for a single insertion of the probe, in contrast to the dual-probe system and method known in the prior art, thereby minimizing potential damage to the nervous system. As such, the present invention provides a disposable electrode that permits precise gene delivery into nervous tissue that does not require viral vectors.

### BRIEF DESCRIPTION OF THE DRAWINGS

For a fuller understanding of the invention, reference should be made to the following detailed description, taken in connection with the accompanying drawings, in which:

FIG. 1 is a diagrammatic view of the electrode assembly in accordance with the present invention.

### DETAILED DESCRIPTION OF THE PREFERRED EMBODIMENT

In the following detailed description of the preferred embodiments, reference is made to the accompanying drawings, which form a part hereof, and within which are shown by way of illustration specific embodiments by which the invention may be practiced. It is to be understood that other embodiments may be utilized and structural changes may be made without departing from the scope of the invention.

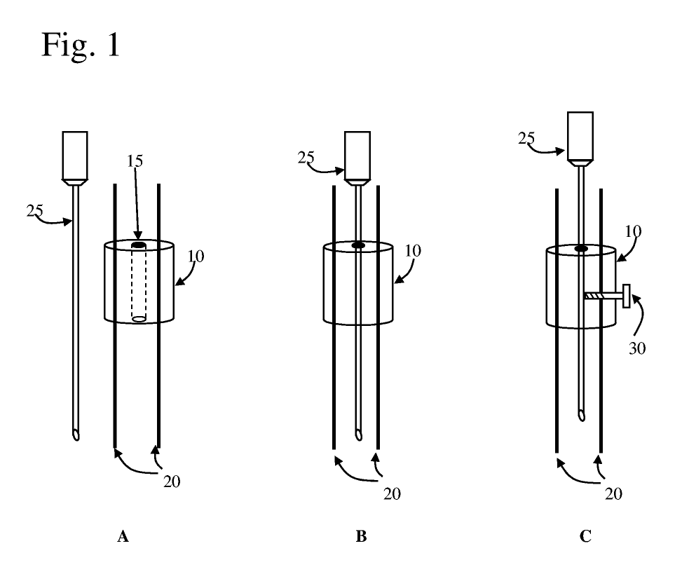

According to a particular embodiment, the electrode assembly is designed for application for brain tissue, as shown with reference to FIG. 1. The device consists of an injection cannula 25 and a pair of electrodes 20. The electrodes 20 are permanently held at a specific distance apart by the cast polymer cylinder 10. In an exemplary embodiment, the electrodes are separated by a distance of 1 mm. However, other dimensional specifics are within the scope of the invention. In the center of the cast polymer cylinder is a channel 15 through which the injection cannula 25 is inserted and held in place by a screw 30 in the insertion guide. The position of the cannula injection probe 25 is variable, thereby allowing for the precise deposition of DNA at selected depths in the tissue as the probe is withdrawn. After deposition of the DNA, a current is delivered between the electrodes, the application of the current sufficient to cause electroporation and delivery of the DNA to the cells of the selected tissue.

It will be seen that the advantages set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention which, as a matter of language, might be said to fall therebetween. Now that the invention has been described,

What is claimed is:

1. A device for the delivery of genes into tissue, the device comprising:
a cast polymer support having an aperture;
at least two spaced apart needle electrodes secured within the cast polymer support and positioned to surround the aperture; and
an axially adjustable injection cannula positioned in the aperture, the axial position of the axially adjustable injection cannula adjustable along the axis of the at least two spaced apart needle electrodes.

2. The device of claim 1, further comprising an injection cannula fastener to secure the injection cannula within the aperture at a selected axial position.

3. The device of claim 1, wherein the cast polymer support is a cast polymer cylinder.

4. The device of claim 1, wherein the at least two space apart needle electrodes are spaced apart by 1 mm.

5. The device of claim 1, further comprising an electric pulse generator for applying pulses to the at least two needle electrodes for electroporation of cells between said electrodes.
